(19) United States
(12) Patent Application Publication
(10) Pub. No.: US 2010/0015242 A1
(43) Pub. Date: Jan. 21, 2010

(54) USE OF A COMPOSITION FOR SKIN TREATMENT UNDER X-IRRADIATION

(75) Inventors: Karin Golz-Berner, Monaco (MC); Leonhard Zastrow, Monaco (MC)

Correspondence Address: Gregory A. Nelson;Novak Druce & Quigg LLP, 525 Okeechobee Blvd, Suite 1500, West Palm Beach, FL, 33401, US

(73) Assignee: COTY PRESTIGE LANCASTER GROUP GMBH, Mainz (DE)

(21) Appl. No.: 12/438,497

(22) PCT Filed: Aug. 24, 2007
(86) PCT No.: PCT/EP07/58832

§ 371 Date: Feb. 23, 2009

(30) Foreign Application Priority Data
Aug. 24, 2006 (DE) 10 2006 040 450.5

(51) Int. Cl. A61K 36/53; A61K 36/752; A61K 36/82; A61K 35/00; A61K 36/537; A61P 17/00

(52) U.S. Cl. 424/520; 424/745; 424/736; 424/729; 424/746

(57) ABSTRACT

The invention relates to the use of a composition in the production of a dermatological agent for skin treatment under repeated X-irradiation. The composition includes oxygen-bearing asymmetrical lamellar aggregates of phospholipids and fluorocarbons as well as specific water-soluble antioxidants, said composition having a radical protection factor of at least 4000×10¹⁴ radicals/mg.

The invention relates to the use of a composition in the production of a dermatological agent for prophylactic or therapeutic treatment of the skin prior to, during and/or subsequent to a therapy with X-rays, especially in the event of previous tumor removal.

The therapeutic use of X-rays is known to result in more or less detrimental side-effects in irradiated skin areas. Especially problematic are aftereffects of breast irradiation following mammary operations, where more or less strong reddening of the skin, hardening of the subcutaneous tissue and significantly reduced elasticity of the skin generally appear. Furthermore, considerable dehydration of the skin can be observed under irradiation. In general, the affected skin areas are treated merely by powdering using commercially available talc-based body powders. The affected females feel alleviation only to a minor extent.

The use of fluorocarbons in dermatological preparations is well-known. EP 91313 describes mixing of perfluorocarbons with therapeutic active substances such as indomethacine, chloramphenicol etc. U.S. Pat. No. 4,569,784 describes a gel with gas transport properties for skin irritations or wounds, which gel consists of fluorocarbon, emulsifier and water.

A dermatic agent is known from EP 0 647 131 B1, which agent contains asymmetrical lamellar aggregates of phospholipids including 30 to 99% phosphatidyl choline and oxygen-loaded fluorocarbons or mixtures of fluorocarbons. The aggregates used therein are capable of penetrating deep into the skin, supplying the skin with oxygen from the outside. Oxygen delivery to undersupplied fatty tissue and deficient supply as a result of arteriosclerosis are mentioned as intended use.

Furthermore, incorporation of antioxidants or radical scavengers in skin preparations is generally known in the fields of beauty culture and dermatology.

The invention is based on the object of improving the restoration of skin functions after use of therapeutic X-irradiation and providing a dermatological agent for that purpose.

According to the invention, a composition for the production of a dermatological agent for prophylactic or therapeutic treatment of the skin under therapy with X-rays is used, said composition comprising:
a) oxygen-bearing asymmetrical lamellar aggregates comprising phospholipids with a phosphatidyl choline content of from 30 to 99 wt. % and fluorocarbons,
b) one or more water-soluble antioxidants selected from the group consisting of white grape peel extract; plant extracts from the Labiatae family; white grapefruit peel extract; green tea extract; green coffee extract; a product obtained by extraction of the bark of Quebracho blanco and subsequent enzymatic hydrolysis and addition of a silkworm extract, in admixture with a gel and phospholipids; and mixtures thereof;
   said composition having a radical protection factor (RPF) of at least 4000×10¹⁴ radicals/mg.

In the sense of the invention, skin treatment “under” therapy with X-rays means that the treatment is effected prior to irradiation, during irradiation, i.e., between single sessions, and/or subsequent to irradiation.

Surprisingly, it was found that a composition of oxygen-bearing asymmetrical lamellar aggregates and specific antioxidants, with the proviso that such a composition in a ready-for-use formulation has a radical protection factor of more than 4000×10¹⁴ radicals/mg, achieves clear improvements when used on skin damaged by X-rays, and, in particular, can significantly improve the elasticity of the skin and bring the skin moisture back to normal condition. The composition, or the dermatological agent, is normally used as a cream, and the effects thereof surpass by far those of powders previously used. Furthermore, despite high shares of aggregates and specific antioxidants, it was found possible to produce stable formulations of aesthetic appearance.

In addition, the dermatological agent includes specific adjuvants and excipients as usually employed in such preparations, e.g. water, preservatives, dyes, thickeners, alcohols, polyols, esters, electrolytes, gelling agents, polar and non-polar oils, polymers, copolymers, emulsifiers, stabilizers and emollients.

It is also possible to add further active substances having a favorable influence on the condition of the skin, e.g. creatine, creatinine, biotin, isoflavones or isoflavonoids. Addition of eucalyptus oil, urea and mineral salts, especially NaCl, marine minerals, osmolytes, vitamin C and derivatives thereof, vitamin A and derivatives thereof, carotenoids and carotenes such as α-carotene, β-carotene, uric acid and derivatives thereof, α-hydroxy acids, tocopheryl acetate is also possible.

A particularly preferred water-soluble antioxidant is a product obtained by extraction of the bark of Quebracho blanco and subsequent enzymatic hydrolysis, which product includes at least 90 wt. % of proanthocyanidin oligomers. In addition, this product includes a silkworm extract which includes the peptide cecropin, amino acids and a vitamin mixture. Both extracts are in the form of a mixture with a gel together with phospholipids, which will be referred to as RPF complex I in the examples.

Other preferred antioxidants are plant extracts of the Labiatae family, including rosemary, oregano, sage and melissa. Particularly preferred are rosemary and oregano, as well as extracts or extract mixtures containing rosmarinic acid, such as the product Origanox®.

In a preferred fashion the antioxidants are present in the range of from 0.5 to 5 wt. %, particularly 2 to 3 wt. %, relative to the weight of the overall composition, the share of a particular antioxidant, such as green tea extract, being 3 wt. % at maximum.

The antioxidants used according to the invention have a radical protection factor of more than 4,000 radicals per mg of substance. In the context with the preferred antioxidant, RPF complex I, details concerning the radical protection factor have been explained in WO 99/66881. The radical protection factor (RPF) determines the activity of binding free radicals by an antioxidant compared to a test substance. The test substance consists of a highly reactive, semi-stable radical which reacts with all known antioxidants. The RPF is measured in such a way that the signal amplitude of the test radical is measured by electron spin resonance (ESR/EPR) before and after being mixed with an antioxidant, and the RPF is calculated therefrom.

The exact measuring method has been described by Herrling, Groth, Fuchs and Zastrow in Conference Materials “Modern Challenges to the Cosmetic Formulation” 5.5.-7-5.97, Dusseldorf, pp. 150-155, Verlag f. chem. Ind. 1997. Starting from the known concentration of test radical or the number of its free radicals (radicals per ml), a signal amplitude S₁ is measured using an ESR spectrometer. Both test radical and antioxidant are dissolved in a water/alcohol solution. Thereafter, the signal amplitude S₂ of the antioxidant is measured. The normalized difference between the two signal amplitudes is the reduction factor RF.

RF=(S₁−S₂)/S₁

The result of the test radical reduction RC×RF is normalized relative to the quantity of product input PI (mg/ml). The radical protection factor is calculated according to the following equation:

$$RPF = \frac{RC [ radicals \text{/} ml ] \times RF}{PI [ mg \text{/} ml ]}$$

The result is RPF=N×10¹⁴ [radicals per mg],

wherein N is a positive real number.

For simplification, the values are given with no “10¹⁴” added.

Preferred ranges for the RPF of the compositions of the present invention are 4000-8000 radicals/mg, particularly 5000-7000 radicals/mg.

The oils used in the invention can be conventional cosmetic oils such as mineral oil; hydrogenated polyisobutene; squalane produced synthetically or from natural products; cosmetic esters or ethers which can be branched or unbranched, saturated or unsaturated; vegetable oils; or mixtures of two or more thereof.

Especially suitable oils are, for example, silicone oils and vegetable oils such as calendula oil, jojoba oil, avocado oil, macadamia nut oil, borage oil, coconut oil, maize oil, cottonseed oil, olive oil, palm nut oil, rapeseed oil, safflower oil, sesame oil, soybean oil, sunflower oil, wheat germ oil, grape seed oil, candlenut oil, thistle oil and mixtures thereof. Especially preferred is borage oil. Preferred silicone oils are cyclic silicone oils such as cyclopentasiloxane, cyclotetrasiloxane and cyclohexasiloxane.

Polyols are also possible components of the dermatological agent according to the invention. For example, such polyols are propylene glycol, dipropylene glycol, ethylene glycol, isoprene glycol, glycerol, butylene glycol, sorbitol and mixtures thereof.

The dermatological agent according to the invention may also include usual humectants such as glycerol, butylene glycol, propylene glycol or mixtures thereof.

The dermatological agent according to the invention may be in the form of a gel. Suitable gelling agents include carbomer, xanthan gum, carrageenan, acacia gum, guar gum, agar, alginates and tyloses, carboxymethylcellulose, hydroxyethylcellulose, quaternized cellulose, quaternized guar, specific polyacrylates, polyvinyl alcohol, polyvinylpyrrolidone, montmorillonite. Preferred is the carbomer gelling agent.

In contrast to usual liposomes representing completely closed lipid bilayer membranes enclosing an aqueous volume, the asymmetrical lamellar aggregates used according to the invention have a three-layer structure. They carry hydrophobic fluorocarbons in their core, which are capable of transporting oxygen. Their interfacial-chemical stabilization is effected primarily by a monolayer with inverse arrangement and secondarily by a structure of bilayers following the former. According to the invention, the asymmetrical lamellar aggregates therefore always have at least one three-layer structure, in contrast to the known bilayer vesicles. Owing to the peculiarity of their structural arrangement, they are referred to as asymmetrical lamellar aggregates. Their exceptional colloid-chemical stability is presumably based on the lamellar structure and the surface charge of the aggregates. The latter is to be attributed to the selection of suitable phospholipids or their mixtures of natural and synthetic origin. Primarily, phospholipids, particularly phosphatidyl choline in the quoted concentration range of from 30 to 99% optionally in combination with lysolecithins at a concentration of from 0.1 to 10% and/or charged phospholipids in the concentration range of from 0.1 to 30 wt. % are responsible for an advantageous effect in this sense. The quoted effect of the phospholipids is verified by corresponding negative zeta potentials and by the measurement of charge densities (when titrated with a cationic polyelectrolyte).

The share of asymmetrical lamellar aggregates is preferably in the range of from 8 to 30 wt. %, especially 10 to 25 wt. %, the share of perfluorocarbons, preferably perfluorodecaline, in the aggregates being 10 to 45 wt. %, preferably 20 to 40 wt. %, relative to the overall weight of the aggregates.

As mentioned above, a preferred embodiment of the composition or dermatological agent according to the invention is a cream. However, the dermatological agent can also be in the form of a lotion or ointment. Surprisingly, the combination of oxygen-bearing aggregates and specific antioxidants shows significantly higher effectiveness compared to the single components. Thus, it was possible to determine in comparative tests that skin moisture and skin elasticity are improved to an unexpected extent.

The present invention is also directed to a method for the prophylactic or therapeutic treatment of a subject's skin under X-ray therapy, which method is characterized in that, prior to beginning the treatment and following repeated treatments, a dermatological agent is applied to the skin areas exposed to the X-rays, which agent comprises a composition comprising:
a) oxygen-bearing asymmetrical lamellar aggregates comprising phospholipids with a phosphatidyl choline content of from 30 to 99 wt. % and fluorocarbons,
b) one or more water-soluble antioxidants selected from the group consisting of white grape peel extract; plant extracts from the Labiatae family; white grapefruit peel extract; green tea extract; green coffee extract; a product obtained by extraction of the bark of Quebracho blanco and subsequent enzymatic hydrolysis and addition of a silkworm extract, in admixture with a gel and phospholipids; and mixtures thereof;
   said agent having a radical protection factor (RPF) of at least 4000×10¹⁴ radicals/mg.

The invention will be explained in greater detail below with reference to examples. All the details are given in weight percent unless otherwise stated.

### EXAMPLE 1

### Body Cream I

| | |
|---|---|
| Range | preferred |

Phase A

| | |
|---|---|
| Water | q.s. ad 100 |

| | | |
|---|---|---|
| Glycerol | 2-5 | 2 |
| Carbomers | 0.3-0.5 | 0.3 |
| 2-Bromo-2-nitropropane-1,3-diol | 0.08-1.5 | 0.1 |
| Tetrasodium EDTA | 0.01-0.04 | 0.04 |

Phase B

| | | |
|---|---|---|
| Isopropyl myristate | 1.5-3 | 3 |
| PPG-15 stearyl ether + BHT | 3-4.5 | 3 |
| Shea butter | 3-5 | 3 |
| Steareth-2 | 1-3 | 2.3 |
| Tocopheryl acetate | 1-5 | 2 |
| Borago officinalis seed oil | 0.5-2 | 2 |
| Cyclopentasiloxane | 0.5-2.0 | 1.9 |
| Steareth-21 | 0.5-2 | 1.5 |
| Cyclohexasiloxane | 0.3-1.5 | 1 |
| Cyclotetrasiloxane | 0.02-0.03 | 0.025 |

Phase C

| | | |
|---|---|---|
| Magnesium aluminum silicate | 0.8-1.2 | 1 |
| Xanthan gum | 0.1-0.6 | 0.6 |
| Panthenol & water | 0.1-1 | 0.5 |

Phase D

| | | |
|---|---|---|
| RPF complex I* | 0.5-5 | 2 |
| AOCS** | 5-25 | 10 |
| Green tea extract | 0.5-4.8 | 3 |

*According to WO 99/66881, active substance complex. To produce said active substance complex, a gel base was initially produced. To this end, a gel powder such as carbomer was added to water, homogenized therein and subsequently neutralized with triethanolamine, for example. Thereafter, ethanol and glycerol were added to improve the incorporability, and the mixture was well stirred.
To this gel base, a mixture of phospholipids (Phoslipon ®), Quebracho extract and silkworm extract was added and mixed at a temperature of 45° C. at maximum. Thereafter, another portion of the above gel or a second gel such as guar propyltriammonium chloride was added and stirred well with the whole mixture at elevated temperature, but below 45° C. In this way, the active substance complex referred to as RPF complex I was obtained.
**Produced from 40% perfluorodecaline, 4% Lecithin MK 05, 6.4% PPG, 0.1% Bronopol ® and 33.5% water by adding glycerol and PPG to the Lecithin at <40° C., adding perfluorodecaline in small amounts at the same temperature and moderate number of revolutions (500-1000 rpm) of the stirrer and finally adding water and Bronopol in small amounts under identical conditions followed by increasing the number of revolutions up to 3000 rpm and subsequently lowering the temperature and stirring to homogeneity.

The production of the composition of Example 1 proceeds via separate production of phases A and B at about 75° C. with stirring. The phases A and B are combined and homogenized for about 20 minutes, followed by cooling to 40° C. Phase C is added with stirring, and phase D is added with stirring at about 35° C. to yield a formulation which is stable for months and has an aesthetic, slightly yellowish appearance.

The RPF of the preferred composition is measured to be 5800 radicals/mg.

### EXAMPLE 2

### Body Cream II

The composition corresponded to that of Example 1, but included 25% AOCS in phase D.

### EXAMPLE 3

### Body Cream III

Phase A

| Water | q.s. ad 100 |
|---|---|
| Glycerol | 2 |
| Carbomer | 0.3 |
| 2-Bromo-2-nitropropane-1,3-diol | 0.1 |
| Tetrasodium EDTA | 0.03 |

Phase B

| | |
|---|---|
| Isopropyl myristate | 3 |
| PPG-15 stearyl ether + BHT | 2.8 |
| Shea butter | 3 |
| Steareth-2 | 2.4 |
| Tocopheryl acetate | 1.8 |
| Borago officinalis seed oil | 2 |
| Cyclopentasiloxane | 2 |
| Steareth-21 | 1.5 |
| Cyclohexasiloxane | 1.2 |
| Cyclotetrasiloxane | 0.03 |

Phase C

| | |
|---|---|
| Magnesium aluminum silicate | 1 |
| Xanthan gum | 0.6 |
| Panthenol & water | 0.5 |

Phase D

| | |
|---|---|
| AOCS** | 25 |
| Green tea extract | 2 |
| RPF complex I | 1 |

**Produced as in Example 1.

Production and appearance of the composition corresponded to that of Example 1. The RPF was measured to be 4900 radicals/mg s.

### EXAMPLE 4

40 female patients (45-75 years of age) having undergone breast-conserving operation of a mammary carcinoma were included in a randomized double-blind study with placebo control. Therapy was started 1 week prior to beginning a subsequent treatment with X-rays and continued for 5 weeks during irradiation, i.e., between single irradiations.

Irradiation in 25 treatments using 2 Gy each time, totalling 50 Gy (instrument: Mevatron Primus®).

Amount of O/W cream applied: 10 mg/cm² skin surface twice a day on the irradiated areas of the breast.

Verum group: formulation in accordance with Example 1.

Placebo group: formulation in accordance with Example 1 including no phase D.

Assessment:

1. Assessment of skin reddening using score units 0-4:
0=negative, no irritation
1=mild erythema, visible, pink-colored
2=erythema, pale-red
3=erythema (livid)
4=strong erythema with papulas/pustulas

The score assessment shows a significant increase in the placebo group as early as in the third week, while the verum group is free of irritations at that point. The score finding significantly increases in the placebo group and only moderately in the verum group. After 5 weeks the verum group shows an irritation score under radiation treatment which is about 40% lower than the placebo group score.

2. Skin moisture measurement using the Corneometer® 820

After a one-week pretreatment of about 93 relative units (RU), the values in both groups dropped down to about 83 RU (verum) and 76 RU (placebo), respectively, showing a difference of about 15% with 73 RU (verum) and 62 RU (placebo) after 5 weeks.

3. Skin elasticity measurement using the Cutometer® SEM 575 (viscoelasticity and plasticity)

The elasticity index in the verum group showed a virtually continuous increase from 2 to 26 in the course of 5 weeks, while the increase in the placebo group was only from 2 to 8, so that the verum group was superior to the placebo group by 19%.

In total, the study showed a clear improvement in all 3 assessed criteria for the irradiated skin areas of those patients who had used a cream of the present invention. The values of the verum group were confirmed in follow-up examinations after 3 months, then 6 months and up to 3 years.

### EXAMPLE 5

Measurements on tissue samples were carried out to clarify the influence of antioxidants and oxygen-bearing aggregates, wherein skin elasticity and skin moisture were assessed according to the method described in Example 4.

Amount applied: 2 mg/cm² at 21° C. and 52% relative humidity Corneometer measurement after 0.5 h-2 h-8 h following single application; figures in % relative to the initial value.

Cutometer measurement after 3 days-6 days-14 days following application once a day; figures in % increase of the ratio Uᵥ/Uₑ (viscoelastic return after elastic deformation (vacuum)).

Cream A: cream in accordance with Example 2

Cream B: cream in accordance with Example 2 without 2% RPF complex I and 3% green tea extract

Cream C: cream in accordance with Example 2 without 25% AOCS

| | A | B | C | |
|---|---|---|---|---|
| Elasticity | 12-19-24 | 8-12-16 | 4-6-6 | (0.5-2-8 h) |
| Moisture | 92-88-86 | 88-76-59 | 74-41-15 | (3-6-14 d) |

As can be seen, the cream according to the invention shows significantly higher values wherein synergism can be recognized.

### EXAMPLE 6

### Body Cream IV

The compositions of phases A, B and C correspond to those of Example 1.

| Phase D | Range | Preferred |
|---|---|---|
| RPF complex I * | 0.5-5 | 2.5 |
| AOCS ** | 5-25 | 15 |
| Green tea extract | 0.5-4.8 | 3 |
| Rosmarinus officinalis | 0.1-0.5 | 0.1 |
| flower extract | | |

* and ** see Example 1.

The RPF for the preferred composition is measured to be 5800 radicals/mg.

### EXAMPLE 7

### Body Cream V

The composition corresponds to that of Example 6. Additionally included is Oreganum vulgare leaf extract (range: 0.1-5%; preferred: 1%). The RPF for the preferred composition is measured to be 6820 radicals/mg.

### EXAMPLE 8

### Body Cream VI

The compositions of phases A, B and C correspond to those of Example 1.

| Phase D | Range | Preferred |
|---|---|---|
| RPF complex I * | 0.5-5 | 2.8 |
| AOCS ** | 5-25 | 17 |
| Green tea extract | 0.5-4.8 | 2.8 |
| Sea water | 0.2-0.5 | 0.2 |
| Vitis vinifera (white grape) | 0.5-1 | 0.5 |
| skin extract (Quest Int., USA/ | | |
| Givaudan-Roure, USA) | | |

* and ** see Example 1.

The RPF for the preferred composition is measured to be radicals/mg.

### EXAMPLE 9

### Body Cream VII

The compositions of phases A, B and C correspond to those of Example 1.

| Phase D | Range | Preferred |
|---|---|---|
| RPF complex I * | 0.5-5 | 2.3 |
| AOCS ** | 5-25 | 13 |
| Salvia officinalis extract | 0.1-5 | 1.2 |

* and ** see Example 1.

The RPF for the preferred composition is measured to be radicals/mg.

### EXAMPLE 10

### Body Cream VIII

The composition corresponds to that of Example 9. Additionally included is Citrus grandis (grapefruit) skin extract (range: 0.1-3; preferred: 2.0). The RPF for the preferred composition is measured to be 5120 radicals/mg.

### EXAMPLE 11

### Body Cream IX

The composition corresponds to that of Example 1 without green tea extract. The RPF is measured to be 5240 radicals/mg.

### EXAMPLE 12

### Body Cream X

The composition corresponds to that of Example 6 without green tea extract. The RPF is measured to be 5330 radicals/mg.

1. A method of prophylactic or therapeutic treatment of the skin under therapy with X-rays, said method comprising:
applying a dermatological agent to skin under therapy with X-rays, said dermatological agent comprising a composition comprising:
a) oxygen-bearing asymmetrical lamellar aggregates comprising phospholipids with a phosphatidyl choline content of from 30 to 99 wt. % and fluorocarbons,
b) one or more water-soluble antioxidants selected from the group consisting of plant extracts from the Labiatae family; white grape peel extract; white grapefruit peel extract; green tea extract; green coffee extract; a product obtained by extraction of the bark of Quebracho blanco and subsequent enzymatic hydrolysis and addition of a silkworm extract, in admixture with a gel and phospholipids; and mixtures thereof;
said composition having a radical protection factor (RPF) of at least 4000×10¹⁴ radicals/mg.

2. The method of prophylactic or therapeutic treatment according to claim 1, characterized in that the share of asymmetrical lamellar aggregates ranges from 8 to 30 wt. %, relative to the overall weight of the composition.

3. The method of prophylactic or therapeutic treatment according to claim 1, characterized in that the share of antioxidants ranges from 0.5 to 5 wt. %, relative to the overall weight of the composition, preferably from 2 to 3 wt. %.

4. The method of prophylactic or therapeutic treatment according to claim 1, characterized in that the plant extracts of the Labiatae family are selected among extracts of rosemary, oregano, sage and melissa, with rosemary and oregano being particularly preferred.

5. The method of prophylactic or therapeutic treatment according to claim 1, characterized in that the antioxidant is a product obtained by extraction of the bark of Quebracho blanco and subsequent enzymatic hydrolysis and addition of a silkworm extract, in admixture with a gel and phospholipids.

6. The method of prophylactic or therapeutic treatment according to claim 1, characterized in that the asymmetrical lamellar aggregates have a structure with at least three layers.

7. The method of prophylactic or therapeutic treatment according to claim 1, characterized in that the asymmetrical lamellar aggregates include 20 to 45 wt. % perfluorodecaline, relative to the overall weight of the aggregates.

8. A dermatological agent comprising a composition, characterized by
a) oxygen-bearing asymmetrical lamellar aggregates comprising phospholipids with a phosphatidyl choline content of from 30 to 99 wt. % and fluorocarbons,
b) one or more water-soluble antioxidants selected from the group consisting of plant extracts from the Labiatae family; white grape peel extract; white grapefruit peel extract; green tea extract; green coffee extract; a product obtained by extraction of the bark of Quebracho blanco and subsequent enzymatic hydrolysis and addition of a silkworm extract, in admixture with a gel and phospholipids; and mixtures thereof;
said composition having a radical protection factor (RPF) of at least 4000×10¹⁴ radicals/mg.
